(19) United States
(12) Patent Application Publication
(10) Pub. No.: US 2008/0255354 A1
(43) Pub. Date: Oct. 16, 2008

(54) Method for Producing Alkoxysilyl Methyl Isocyanurates

(75) Inventors: Alfred Popp, Unterhaching (DE); Klaus Stowischek, Munchen (DE)

Correspondence Address: BROOKS KUSHMAN P.C., 1000 TOWN CENTER, TWENTY-SECOND FLOOR, SOUTHFIELD, MI, 48075, US

(73) Assignee: Wacker Chemie AG, Munich (DE)

(21) Appl. No.: 11/817,440

(22) PCT Filed: Mar. 3, 2006
(86) PCT No.: PCT/EP2006/001969

§ 371 Date: Aug. 30, 2007

(30) Foreign Application Priority Data
Mar. 3, 2005 (DE) 10 2005 009 790.1

(51) Int. Cl. C07D 251/30

(52) U.S. Cl. 544/221

(57) ABSTRACT

Alkoxysilylmethyl isocyanurates are prepared in high space time yield by reaction of a chloromethylalkoxysilane with a metal isocyanate in the presence of a tetrakis[hydrocarbon]ammonium phase transfer catalyst, with minimal formation of byproducts.

The invention relates to a method for producing alkoxysilylmethyl isocyanurates.

Alkoxysilylalkyl isocyanurates are used as accelerators or promoters for the adhesion of room temperature-crosslinking organosiloxanes and silane-modified polymers, as an additive for organosiloxane mixtures in the coating of fibers or in automotive finishes. The most important members of this class of substances are the alkoxysilylpropyl isocyanurates, which have been produced industrially for more than 30 years. Alkoxysilylmethyl isocyanurates have considerable advantages compared with said alkoxysilylpropyl isocyanurates. Owing to the spatial closeness of the alkoxysilyl group to the isocyanurate ring, the alkoxy groups are activated so that a considerable increase in reactivity (crosslinking reaction) is observable in comparison with the propyl isocyanurates.

Alkoxysilylmethyl isocyanurates are known. The only explicit mention of these compounds occurs in JP 57030336 A2. There, the use thereof for the surface coating of semiconductors is claimed. However, there has to date been no information about a suitable production method.

In addition, alkoxysilylalkyl isocyanurates and methods for the production thereof are generally known. Thus, U.S. Pat. No. 3,821,218 A describes alkoxysilylalkyl isocyanurates, the alkyl spacers being selected from the group consisting of C₁-C₈-alkyl. However, there are examples of only propyl spacers (C₃-alkyl), the uncatalyzed reaction of chloropropylalkoxysilanes with metal isocyanates in DMF being used for the production thereof. U.S. Pat. No. 3,494,951 A, U.S. Pat. No. 3,598,852 A, U.S. Pat. No. 3,607,901 A and DE 2419125 A describe analogous methods. U.S. Pat. No. 5,218,133 A describes the preparation of silyl isocyanurates by a) reaction of an aminosilane with dialkyl or diaryl carbonates, b) neutralization and c) subsequent thermal reaction of the resulting carbamate in the presence of a crack catalyst.

U.S. Pat. No. 5,905,150 A demonstrates, on the basis of the reaction of alkoxysilylpropyl chlorides with metal isocyanates, that methods using phase-transfer catalysts based on guanidinium salts are suitable for the synthesis of corresponding isocyanurates. MacGregor et al. subsequently showed that exclusively guanidinium salt phase-transfer catalysts are suitable for this system (Polymer Preprints, 2001, 42(1), pages 167-168).

With the use of tetraethylammonium iodide as a phase-transfer catalyst, on the other hand, the preferred formation of the corresponding isocyanatoalkylsilanes and uretdiones is described for the reaction of ω-haloalkylsilanes (e.g. chloromethyltrimethylsilane) with KOCN (Smetankina et al., Zhurnal Obschchei Khimii (1969), 39(9), 2016-20). The occurrence of isocyanurates was not observed.

The invention relates to a method for producing alkoxysilylmethyl isocyanurates of the general formula I

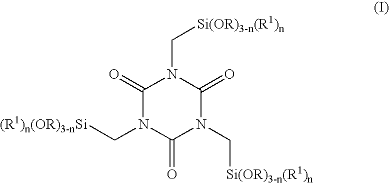

in which chloromethylalkoxysilanes of the general formula II

(RO)₃₋ₙ(R¹)ₙSi—CH₂—Cl (II)

are reacted with metal isocyanates of the general formula III

M(OCN)ₘ (III)

in the presence of tetrahydrocarbonammonium salt catalysts of the general formula IV

(R²)₄N⁺X⁻ (IV)

in which
R is a C₁-C₁₅-hydrocarbon radical or acetyl radical,
R¹ is a hydrogen atom or a Si—C bonded C₁-C₂₀-hydrocarbon radical which is optionally substituted by —CN, —NCO, —NR³₂, —COOH, —COOR³, -halogen, -acryloyl, -epoxy, —SH, —OH or —CONR³₂ and in which non-neighboring methylene units may be replaced by —O—, —CO—, —COO—, —OCO— or —OCOO—, —S—, or —NR³— groups and in which one or more, non-neighboring methine units may be replaced by —N═, —N═N— or —P═ groups,
n has the value 0, 1 or 2,
M is an alkali metal or alkaline earth metal
m has the value 1 or 2,
R² is a C₁-C₂₀-hydrocarbon radical optionally substituted by —CN, —OH or halogen,
R³ is a hydrogen atom or a C₁-C₂₀-hydrocarbon radical which is optionally substituted by —CN, halogen, —SH or —OH and in which non-neighboring methylene units may be replaced by —O—, —CO—, —COO—, —OCO— or —OCOO— or —S— groups, and
X is a radical selected from OH, F, Cl, Br, I, ClO₄, NO₃, BF₄, AsF₆, BPh₄, PF₆, AlCl₄, CF₃SO₃, HSO₄ and SCN.

If the prior art is summarized, the person skilled in the art must assume that, for the synthesis of alkoxysilylmethyl isocyanurates, the use of tetrahydrocarbonammonium salt phase-transfer catalysts results in no improvements of the production process but, on the contrary, leads to a change in the product spectrum, namely to the formation of uretdiones.

It has now been found that the abovementioned assumptions cannot be applied to the preparation of alkoxysilylmethyl isocyanurates. The use of tetrahydrocarbonammonium salt PT catalysts leads to a considerable improvement in the reaction performance, in particular an increase in the reaction rate, increase in the space-time performance and reduced formation of byproducts.

Preferably, R is a C₁-C₈-hydrocarbon radical, particularly preferably a C₁-C₃-alkyl radical, in particular methyl or ethyl radical.

Preferably, R¹ is a C₁-C₈-hydrocarbon radical, particularly preferably a C₁-C₃-alkyl radical or a phenyl radical, in particular a methyl or ethyl radical.

Preferably, M is selected from Li, Na, K, Rb, Be, Mg, Ca, Sr and Ba. Particularly preferred examples of suitable metal isocyanates of the general formula III are potassium and sodium isocyanate.

Preferably, R² is a linear or branched aliphatic C₁-C₁₂-hydrocarbon radical or a C₁-C₁₂-hydrocarbon radical aliphatically bonded to the nitrogen atom, particularly preferably a linear aliphatic C₁-C₈-hydrocarbon radical.

Preferably, R³ is a C₁-C₁₂-hydrocarbon radical, particularly preferably a C₁-C₆-alkyl radical, in particular a methyl or ethyl radical.

Preferably, X is selected from Cl, Br, I, BF₄ and BPh₄, particularly preferably from I and BF₄.

Examples of suitable chloromethylalkoxysilanes of the general formula II include chloromethylmethoxydimethylsilane, chloromethyldimethoxymethylsilane, chloromethyltrimethoxysilane, chloromethylethoxydimethylsilane, chloromethyldiethoxymethylsilane, chloromethyltriethoxysilane, chloromethylacetoxydimethylsilane, chloromethyldiacetoxymethylsilane and chloromethyltriacetoxysilane. Particularly preferred chloromethylalkoxysilanes are chloromethylmethoxydimethylsilane, chloromethyldimethoxymethylsilane and chloromethyltrimethoxysilane.

Examples of suitable tetrahydrocarbonammonium salt catalysts of the general formula IV include tetramethylammonium iodide, tetraethylammonium iodide, tetrabutylammonium iodide, benzyltributylammonium chloride, tetrabutylammonium bromide, tetraethylammonium tetrafluoroborate, tetrabutyl-ammonium tetrafluoroborate and tetrabutylammonium tetraphenyloborate. Particularly preferred tetrahydrocarbonammonium salt catalysts are tetrabutylammonium iodide, tetraethylammonium iodide, tetramethylammonium iodide and tetrabutylammonium tetrafluoroborate and mixtures thereof.

The method according to the invention can be carried out in the presence and absence of a solvent. The method preferably takes place in a solvent or solvent mixture. Polar aprotic solvents which do not influence the reaction in an undesired manner or lead to undesired secondary reactions are preferably used as the solvent or solvent mixture. Suitable solvents are all those in which the compounds used are at least partially soluble under operating conditions with regard to concentration and temperature. Dimethylformamide or solvent mixtures which contain dimethylformamide is or are particularly suitable as the solvent. Preferred solvents or solvent mixtures are those whose boiling point or boiling range is not more than 200° C. at 0.1 MPa.

The addition of the reactants can be effected either batchwise or continuously. In a preferred embodiment, a reactant of the general formula II and/or III is metered in. Advantageously, the metal isocyanate and the tetrahydrocarbonammonium salt catalyst are initially introduced, optionally in the desired solvent, and the chloromethylalkoxysilane is metered in at the suitable process temperature.

The reaction is advantageously carried out at a temperature of from 0° C. to +200° C., preferably from +80° C. to +160° C.

Preferably, from 0.8 to 1.5 mol, in particular from 1 to 1.2 mol, of metal isocyanates of the general formula III are used per 1 mol of chloromethylalkoxysilanes of the general formula II. Preferably, from 0.1 to 200 mmol, in particular from 1 to 50 mmol, of tetrahydrocarbonammonium salt catalyst of the general formula IV are used per 1 mol of chloromethylalkoxysilanes of the general formula II.

The course of the reaction can be easily monitored by customary methods, such as, for example, by means of GC or HPLC.

All above symbols of the above formulae have their meanings in each case independently of one another.

In the following examples, all stated amounts and percentages are based on weight, all pressures are 0.10 MPa (abs.) and all temperatures are 20° C., unless stated otherwise in each case.

### EXAMPLE 1

### Preparation of 1,3,5-tris[(methoxydimethylsilanyl)methyl]-[1,3,5]triazinane-2,4,6-trione

The apparatus is flushed with argon for about 30 min. DMF (87.9 g, 1.20 mol), NaOCN (21.5 g, 0.33 mol), chloromethylmethoxydimethylsilane (41.1 g, 0.30 mol) and 1.6 mol % of tetramethylammonium iodide (1.0 g, 5.0 mmol) are then initially introduced at room temperature. Heating is effected to 130° C. in the course of one hour and this temperature is maintained for a further two hours. After cooling to room temperature, the reaction mass is filtered and the solvent is removed under reduced pressure. The residues have the following compositions (gas chromatographic determination of the peak area percentage: GC_{PA-%}):

| | |
|---|---|
| Isocyanurate: 1,3,5-tris[(methoxydimethylsilanyl)methyl]- | 87.0% |
| [1,3,5]triazinane-2,4,6-trione | |
| Uretdione: 1,3-bis[(methoxydimethylsilanyl)methyl]- | 0.5% |
| [1,3]diazetidine-2,4-dione | |
| Allophanate: methyl | 0.4% |
| [(methoxydimethylsilyl)methyl][[[(methoxydimethylsilyl)- | |
| methyl]-amino]carbonyl]carbamate | |
| Methylsilyl carbamate: methyl | 1.4% |
| bis[(methoxydimethylsilanyl)methyl]carbamate | |
| Carbamate: methyl[(methoxydimethylsilanyl)methyl]carbamate | 6.2% |

The clear colorless isocyanurate is separated off by distillation and purified (b.p. 145° C./0.03 mbar): ¹H-NMR (500 MHz, CDCl₃):δ 3.48, 0.21 ppm.

¹³C-NMR (75 MHz, CDCl₃): δ 148.8, 50.2, 32.7, −2.85 ppm.

²⁹Si-NMR (60 MHz, CDCl₃): δ 14.43 ppm.

### COMPARATIVE EXAMPLE 1

### Not According to the Invention

### Preparation of 1,3,5-tris[(methoxydimethylsilanyl)methyl]-[1,3,5]triazinane-2,4,6-trione

The experimental procedure is analogous to example 1, but without the tetrahydrocarbonammonium salt catalyst. In this variant, the reaction mass has the following composition (GC_{PA-%}):

| | |
|---|---|
| Isocyanurate: 1,3,5-tris[(methoxydimethylsilanyl)methyl]- | 3.0% |
| [1,3,5]triazinane-2,4,6-trione | |
| Uretdione: 1,3-bis[(methoxydimethylsilanyl)methyl]- | 27.0% |
| [1,3]diazetidine-2,4-dione | |
| Allophanate: methyl | 2.5% |
| [(methoxydimethylsilyl)methyl][[[(methoxydimethylsilyl)- | |
| methyl] amino]carbonyl]carbamate | |
| Methylsilyl carbamate: methyl | 1.9% |
| bis[(methoxydimethylsilanyl)methyl]carbamate | |
| Carbamate: methyl[(methoxydimethylsilanyl)methyl]carbamate | 3.8% |
| Starting material: chloromethylmethoxydimethylsilane | 62.0% |

### EXAMPLES 2-7

The examples were carried out analogously to example 1. The amount of tetrahydrocarbonammonium salt is based on mol % relative to chloromethylmethoxydimethylsilane.

| | Tetrahydrocarbonammonium | Amount | GC_{PA-%} |
|---|---|---|---|
| | salt | [mol %] | (isocyanurate) |
| 2 | Tetrabutylammonium | 0.4 | 86.2 |
| | bromide | | |
| 3 | Tetraethylammonium iodide | 0.5 | 85.0 |
| 4 | Tetraethylammonium | 0.5 | 82.6 |
| | tetrafluoroborate | | |
| 5 | Tetrabutylammonium | 0.3 | 82.5 |
| | tetraphenyloborate | | |
| 6 | Tetramethylammonium | 16.7 | 88.7 |
| | iodide | | |
| 7 | Tetramethylammonium | 8.3 | 86.6 |
| | iodide | | |

### EXAMPLE 8

The experimental procedure is analogous to example 1, but with KOCN (26.8 g, 0.33 mol) instead of NaOCN. In this variant, the reaction mass contains 88.3% of isocyanurate [GC_{PA-%}].

### EXAMPLE 9

The experimental procedure is analogous to example 1, but the chloromethylmethoxydimethylsilane is metered in the course of one hour at 130° C. and then stirring is effected for a further 2 h at this temperature.

In this variant, the reaction mass contains 78.8% of isocyanurate [GC_{PA-%}].

### EXAMPLE 10

### Preparation of 1,3,5-tris[(dimethoxymethylsilanyl)methyl]-[1,3,5]triazinane-2,4,6-trione

The experimental procedure is analogous to example 1, but the chloromethyldimethoxymethylsilane is used instead of chloromethylmethoxydimethylsilane.

In this variant, the reaction mass contains 45.0% of isocyanurate [GC_{PA-%}].

### COMPARATIVE EXAMPLE 2

### Not According to the Invention

The experimental procedure is analogous to example 10, but without tetrahydrocarbonammonium salt catalyst.

In this variant, the reaction mass contains 23.0% of isocyanurate [GC_{PA-%}].

The comparison of examples 1 and 9 or comparative examples 1 and 2 shows a considerably greater reaction rate with the use, according to the invention, of a phase-transfer catalyst. In comparison with the uncatalyzed reaction, the use, according to the invention, of a phase-transfer catalyst permits virtually a doubling of the space-time performance in the comparison of the product formation of example 1 (˜0.34 mol L⁻¹ h⁻¹) with example 4 in U.S. Pat. No. 3,821,218 A (˜0.13 mol L⁻¹ h⁻¹)).

1. A method for producing alkoxysilylmethyl isocyanurates of the formula I 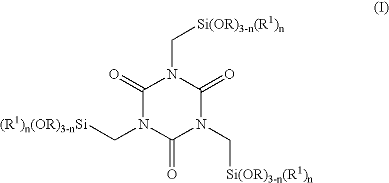
comprising reacting chloromethylalkoxysilanes of the formula II
(RO)₃₋ₙ(R¹)ₙSi—CH₂—Cl (II)
with metal isocyanates of the formula III
M(OCN)ₘ (III)
in the presence of tetrakis[hydrocarbon]ammonium salt catalysts of the formula IV
(R²)₄N⁺X⁻ (IV)
in which
R is a C₁-C₁₅-hydrocarbon radical or acetyl radical,
R¹ is a hydrogen atom or an Si—C bonded C₁-C₂₀-hydrocarbon radical which is optionally substituted by —CN, —NCO, —NR³₂, —COOH, —COOR³, -halogen, -acryloyl, -epoxy, —SH, —OH or —CONR³₂ and in which non-neighboring methylene units are optionally replaced by —O—, —CO—, —COO—, —OCO— or —OCOO—, —S—, or —NR³— groups, and in which one or more, non-neighboring methine units are optionally replaced by —N═, —N═N— or —P═ groups,
n is 0, 1 or 2,
M is an alkali metal or alkaline earth metal
m is 1 or 2,
R² is a C₁-C₂₀-hydrocarbon radical optionally substituted by —CN, —OH or halogen,
R³ is a hydrogen atom or a C₁-C₂₀-hydrocarbon radical which is optionally substituted by —CN, halogen, —SH or —OH and in which non-neighboring methylene units may be replaced by —O—, —CO—, —COO—, —OCO— or —OCOO— or —S— groups, and
X is an OH, F, Cl, Br, I, ClO₄, NO₃, BF₄, AsF₆, BPh₄, PF₆, AlCl₄, CF₃SO₃, HSO₄, or SCN radical.

2. The method of claim 1, wherein the chloromethylalkoxysilane of formula II comprises chloromethylmethoxydimethylsilane, chloromethyldimethoxymethylsilane or chloromethyltrimethoxysilane.

3. The method of claim 1, wherein the metal isocyanate of formula III comprises potassium isocyanate or sodium isocyanate.

4. The method of claim 1, wherein the tetrakis[hydrocarbon]ammonium salt catalyst of formula IV comprises tetrabutylammonium iodide, tetraethylammonium iodide, tetramethylammonium iodide or tetrabutylammonium tetrafluoroborate, or mixtures thereof.

5. The method of claim 1, wherein dimethylformamide or a solvent mixture comprising dimethylformamide is used as a solvent for the reaction.

6. The method of claim 1, wherein the reaction is carried out at a temperature of from +80° C. to +160° C.

7. The method of claim 1, wherein the reaction is a batch reaction.

8. The method of claim 1, wherein the metal isocyanate of formula III and tetrakis[hydrocarbon]ammonium salt catalyst of formula IV are initially introduced and the chloromethylalkoxysilane of formula II is metered in.
